(19) United States
(12) Patent Application Publication
(10) Pub. No.: US 2021/0101009 A1
(43) Pub. Date: Apr. 8, 2021

(54) SYSTEMS AND METHODS FOR ULTRA-FOCAL TRANSCRANIAL MAGNETIC STIMULATION

(71) Applicant: THE GENERAL HOSPITAL CORPORATION, BOSTON, MA (US)

(72) Inventors: Laleh Golestani Rad, Brookline, MA (US); Georgio Bonmassar, Lexington, MA (US); Alvaro Pascual-Leone, Wayland, MA (US)

(21) Appl. No.: 16/971,080

(22) PCT Filed: Feb. 19, 2019
(86) PCT No.: PCT/US19/18634

§ 371 Date: Aug. 19, 2020

Related U.S. Application Data
(60) Provisional application No. 62/632,256, filed on Feb. 19, 2018.

(51) Int. Cl. A61N 1/36; A61N 2/02; A61N 2/00

(52) U.S. Cl. CPC A61N 1/36025; A61N 2/02; A61H 2201/10; A61N 2/006; A61N 2/002

(57) ABSTRACT

A system and method for ultra-focal transcranial magnetic stimulation are provided. In accordance with some aspects of the disclosure, the μTM5 element includes a substrate and, supported by the substrate, microcoils formed using conductive micro-wires having a curved shape and capable of sustaining an electrical signal, wherein the microcoils are configured to produce a focality of stimulation of less than 10 mm when subjected to the electrical signal.

### CROSS-REFERENCE TO RELATED APPLICATIONS

This application is based on, claims priority to, and incorporates herein by reference, in its entirety, U.S. Application Ser. No. 62/632,256 filed on Feb. 19, 2018 and entitled “SYSTEM AND METHOD FOR MICRO-FABRICATED MINIATURIZED COILS FOR ULTRA-FOCAL TRANSCRANIAL MAGNETIC STIMULATION.”

### BACKGROUND

Transcranial magnetic stimulation (TMS) is a non-invasive technique that utilizes magnetic fields to stimulate nerve cells in the brain of a subject. The magnetic fields are generated by electric pulses running through large insulated coils placed over the subject's scalp. In a similar method, transcranial electric stimulation (TES) utilizes scalp electrodes to pass electrical current through specific areas of the brain and alter neuronal excitability. However, unlike TES, TMS does not strongly activate scalp pain fibers. Therefore, longer and more systematic procedures are possible, and with minimal discomfort. Also, TMS can be used to actively identify specific regions of the brain critical for implementing particular cognitive or behavioral functions. Specifically, by altering the activity of brain regions involved with a function of interest, researchers can observe the resulting changes and establish any causal links between the two.

Brief disruption of cortical activity by a TMS pulse applied at the correct time and to the correct brain region, can potentially reveal fundamental insights into the causal chronometry of a given brain area for a given cognitive or behavioral ability. For example, language representation can be studied by inducing speech arrest, or disrupting naming or comprehension. Cortical mapping of language, as well as the systematic exploration of the motor cortical outputs, can also be beneficial for pre-surgical evaluation of patients to help characterize eloquent cortex whilst reducing the need for intraoperative evaluation and are FDA approved. Other uses for TMS mapping include pathophysiology studies and investigations into the evolution of various neurological and psychiatric diseases of the brain. For instance, TMS mapping has been utilized in patients with brain tumors, cerebral palsy, epilepsy, and stroke. More novel clinical applications have also included studies of reduction in intracortical inhibition in neuropathic chronic pain, and studies of altered cortical physiology in basal ganglia-connected areas in patients with Parkinson's disease. Yet another emerging application of TMS is in network stimulation.

As healthy brain function relies on coordinated integration of localized activity across widespread neural networks, understanding how such activity is integrated globally across the brain is arguably one of the greatest challenges facing modern neuroscience. Even though different types of brain stimulation (invasive and non-invasive) are applied in different locations, targets used to treat the same disease most often are nodes in the same brain network. As a result, patient populations treated with invasive techniques (e.g., deep brain stimulation) and TMS have started to converge. Specifically, the potential of therapeutic applications and indeed modulation of specific brain networks have been shown to be effective in the treatment of major depression (for which 6 TMS devices are FDA approved), migraine (one device FDA approved) and Obsessive Compulsive Disorder (one device FDA approved).

Despite great potential, there are critical barriers in further advancement of TMS applications. One major restriction of standard TMS technologies is the limitation in the spatial resolution achievable. As a result, conventional techniques provide reduced focality of stimulation, both in terms of the size of the stimulated area, and the fact that the volume of stimulated tissue does not fall off rapidly. As appreciated, this makes correlating the stimulation site to the targeted cortical region very challenging. More importantly, it makes high spatial resolution and precision impossible. For example it is not possible to separately and specifically target motor and sensory cortices, or separate motor cortical representations

TMS spatial resolution is dictated by the geometry and dimensions of the stimulating coils being utilized, and the physical laws governing them. Therefore, several attempts to increase the spatial resolution of TMS have involved reducing the dimension of the stimulating coils. However, such efforts have had limited success due to practical constraints. For instance, typical TMS coils have a few turns (<30) of the wire winding inside an insulation housing. As the dimension of the coils is reduced (e.g. to diameters less than 2 cm), larger and larger electric currents (few hundreds of kilo amperes) are required to produce sufficient electric fields to produce neural activation in the cortex (˜60-100 V/m). However, passing such large currents through the wires produces excessive amounts of heat and large magnetic forces, which is not tolerated by conventional wires.

Therefore, there is a need for improved TMS technologies that can achieve high resolution and focality, and allow targeting of multiple regions in the central and peripheral nervous system, including closely-spaced regions.

### SUMMARY

The present disclosure overcomes the drawbacks of previous technologies. In accordance with one aspect of the present disclosure, a micro-transcranial magnetic stimulation (μTMS) element for stimulating a subject is provided. The μTMS element includes a substrate and, supported by the substrate, microcoils formed using conductive micro-wires having a curved shape and capable of sustaining an electrical signal, wherein the microcoils are configured to produce a focality of stimulation of less than 10 mm when subjected to the electrical signal.

In accordance with another aspect of the present disclosure, a system for micro-transcranial magnetic stimulation (μTMS) is provided. The system includes a microcoil assembly comprising at least one μTM S element having a focality of stimulation of less than 10 mm, and a signal generator in electrical communication with the microcoil assembly and configured to provide electrical signals to the at least one μTMS element. The system also includes a controller configured to control the signal generator to deliver the electrical signals to the at least one μTMS element, wherein the at least one μTMS element comprises a substrate and, supported by the substrate, microcoils formed using conductive micro-wires having a curved shape and capable of sustaining the electrical signals.

In accordance with yet another aspect of the present disclosure, a method for stimulating a subject is provided. The method includes directing a signal generator, using a controller, to provide an electrical signal to a microcoil assembly having at least one μTMS element. The at least one μTMS element includes a substrate and, supported by the substrate, microcoils formed using conductive micro-wires having a curved shape and capable of sustaining the electrical signal, wherein the at least one μTM S element has a focality of stimulation of less than 10 mm when subjected to the electrical signal.

Features and advantages of the present disclosure will be further apparent from the following description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A is a schematic diagram for an example system, in accordance with aspects of the present disclosure.

FIG. 1B is a schematic diagram for another example system, in accordance with aspects of the present disclosure.

FIG. 2A is a top view of a micro-transcranial magnetic stimulation (μTMS) element, in accordance with aspects of the present disclosure.

FIG. 2B is a side view of one embodiment of the μTMS element in FIG. 2A.

FIG. 2C is a side view of another embodiment of the μTM S element in FIG. 2A.

FIG. 2D is a side view of yet another embodiment of the μTMS element in FIG. 2A.

FIG. 3A is a schematic of a writing diagram for a two-element array of μTM S elements, in accordance with aspects of the present disclosure.

FIG. 3B are graphs showing a ramp shaped pulse for use in cortical stimulation, according to aspects of the present disclosure.

FIG. 4 is an illustration showing an array of μTMS elements, in accordance with aspects of the present disclosure.

FIG. 5 is an illustration showing a method of fabrication of μTMS elements, in accordance with aspects of the present disclosure.

FIG. 6 is a graphical illustration comparing the present stimulation approach in comparison with current state-of-the-art technologies.

FIG. 7 is a flowchart setting forth steps of a process, in accordance with aspects of the present disclosure.

FIG. 8 shows example three-dimensional images of a subject's brain and evoked potentials from an applied stimulation.

FIG. 9 shows cortical output maps, generated in accordance with aspects of the present disclosure.

FIG. 10A is a top view of another μTMS element, in accordance with aspects of the present disclosure.

FIG. 10B is a side view of the embodiment shown in FIG. 10A.

FIG. 10C is another side view of the embodiment shown in FIG. 10A.

FIG. 10D is yet another side view of the embodiment shown in FIG. 10A.

FIG. 10E is another top view of the embodiment shown in FIG. 10A, illustrating a rolling process for producing microcoils, in accordance with aspects of the present disclosure.

FIG. 10F is a top view of the microcoils produced in the process illustrated in FIG. 10E.

FIG. 10G is another top view of the microcoils produced in the process illustrated in FIG. 10E.

FIG. 10H is a side view of one embodiment of the microcoils illustrated in FIG. 10G.

FIG. 10I is a side view of another embodiment of the microcoils illustrated in FIG. 10G.

FIG. 10J is a side view of yet another embodiment of the microcoils illustrated in FIG. 10G, the embodiment including a multilayer stack of microcoils in accordance with aspects of the present disclosure.

FIG. 11 is an image showing stimulation of a subject's peripheral nervous system using a μTMS element, in accordance with aspects of the present disclosure.

The present invention will hereafter be described with reference to the accompanying drawings, wherein like reference numerals denote like elements.

### DETAILED DESCRIPTION

Micro-magnetic stimulation (μMS) is an emerging technology with great promise to revolutionize the noninvasive stimulation of the human brain. Originally developed to directly stimulate single neurons, μMS uses ultra-conductive micro-traces capable of carrying current pulses large enough to elicit neural activation using electromagnetic induction. In this regard, the μMS mechanism of action is similar to that of transcranial magnetic stimulation (TMS). However, because of its ultra-small dimension, μMS induces electric fields with a much higher spatial gradients, rendering it more efficient to elicit focal neural activation.

Recognizing the advantages of μMS technology, the present disclosure herein introduces systems and methods for noninvasive brain stimulation that represents a substantial improvement over the current technology. In particular, a novel design and method of fabrication for micro-transcranial magnetic stimulation (μTMS) elements are provided. As will be described, contemplated μTMS elements may consist of multiple turns of conductive micro-wires or micro-traces carefully formed in a substrate using micro- or nano-fabrication techniques that can provide an unprecedented focality of stimulation. By virtue of their size and novel design, the excitation foci, or activated regions in the subject's central or peripheral nervous system, achievable by the present μTMS elements can be localized to just a few millimeters, which represents a significant improvement as compared to conventional coil technology (1˜5 mm vs. 10˜30 mm).

Previous transcranial magnetic stimulation (TMS) techniques face several challenges, including the inability to stimulate multiple areas simultaneously and the inability to stimulate regions below the cortical surface. By contrast, μTMS elements, in accordance with the present disclosure, can be stacked and combined to form large conformal or multi-location arrays for simultaneous multifocal stimulation. This allows for transcranial brain stimulation that enables noninvasive cortical mapping and multi-focal probing of cortico-cortical interactions. In the realm of network stimulation, simultaneously stimulating multiple sites to inhibit certain nodes while facilitating others is envisioned to introduce a significant leap in the study of altered brain networks in psychiatric and neurological disorders.

Therefore, systems and methods of the present disclosure may be used to generate safe and reliable motor cortical mapping with greater spatial precision than present state-of-the-art technology. For example, it is contemplated interactions between primary somatosensory and primary motor cortices, and separate somatosensory and motor activation of single digits, may be explored using systems and methods of the present disclosure. Other applications may also include transcranial magnetic stimulation for treating neuropsychiatric conditions, as well as other transformative neuroscience research ranging from “virtual lesions” to human neuroplasticity investigations.

In addition, the present stimulation approach can be used to provide a topology that greatly facilitates concurrent application with functional magnetic resonance imaging (fMRI), optical imaging, electroencephalogram (EEG) monitoring, and other applications. Specifically, the small size of μTMS elements described herein allows them to fit inside multi-channel close-fit MRI receiver arrays used to increase fMRI signal-to-noise ratio up to 6 fold at the level of cortical tissue. Also, because the magnetic fields of μTMS elements fall of rapidly, artifacts induced by eddy currents can be significantly reduced for both on MRI images and on EEG electrodes. As such, the possibility of integrating multi-focal μTMS arrays with various monitoring and control systems introduces a revolutionary approach for non-invasive closed-loop stimulation and control.

As appreciated from description herein, in some applications, μTMS elements, by virtue of their reduced size, may be implantable. This would allow continuous/periodic and precise stimulation without requirement of major surgical interventions. Furthermore, the present μTMS elements would be advantageous for stimulating critical anatomical locations, such as the vagus nerve or other nerves.

As described herein, focality may refer to a region or focus in a subject's central or peripheral nervous system where an excitation exceeds a predetermined threshold. The predetermined threshold may be determined based on electric field values, magnetic field values, or gradients thereof, sufficient to achieve a specific response (e.g. a neural activation or inhibition).

In addition, when using terms such as “approximately,” “around” or “about” with reference to a nominal value, a variation of up to +/−20% of the nominal value may be possible.

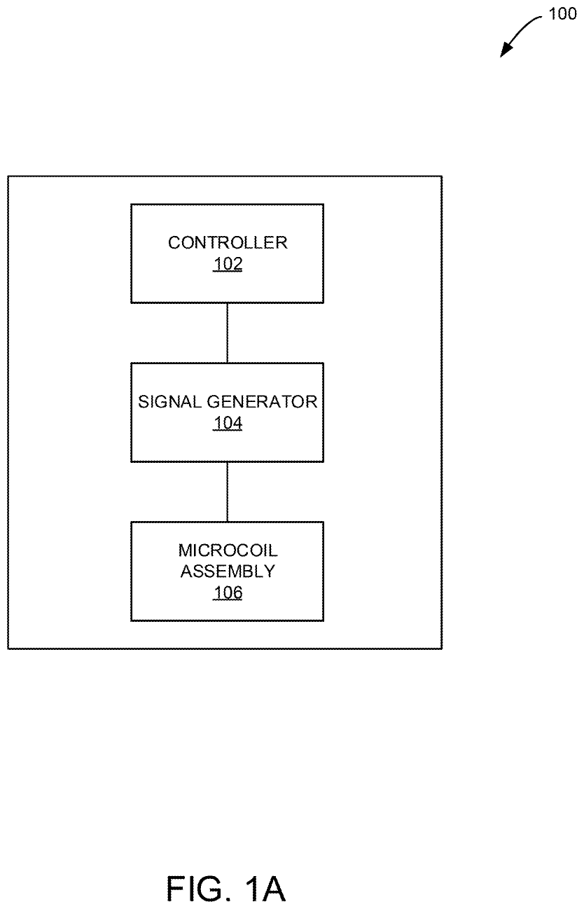

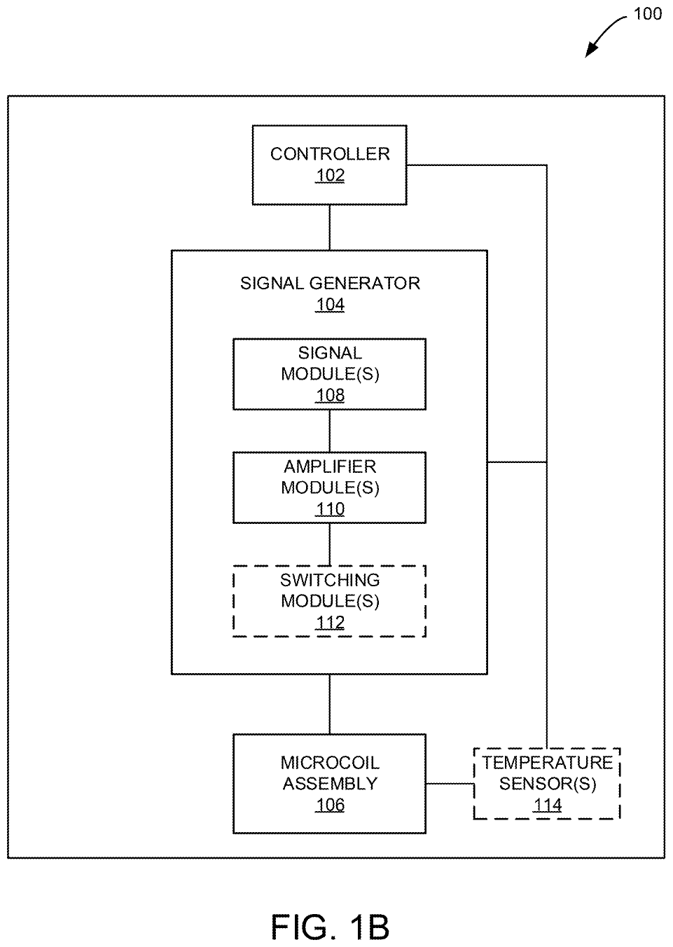

Referring now to FIGS. 1A and 1B, the schematic diagram of an example system 100 for transcranial magnetic stimulation, in accordance with aspects of the present disclosure, is shown. In general, the system 100 may include a controller 102, a signal generator 104, and a microcoil assembly 106 that includes a number of μTMS elements.

The controller 102 is communication with the signal generator 102 and configured to direct the signal generator 102 to provide various signals to the microcoil assembly 106. In some implementations, the controller 102 may be any general-purpose computing system or device, such as a personal computer, workstation, cellular phone, smartphone, laptop, tablet, or the like. As such, the controller 102 may include any suitable hardware and components designed or capable of carrying out a variety of processing and control tasks, including steps for optimizing and directing the signal generator 102 to provide various signals to the microcoil assembly 106. For example, the controller 102 may include a programmable processor or combination of programmable processors, such as central processing units (CPUs), graphics processing units (GPUs), and the like. In some implementations, the controller 102 may be configured to execute instructions stored in a non-transitory computer readable-media. In this regard, the controller 102 may be any device or system designed to integrate a variety of software, hardware, capabilities and functionalities. Alternatively, and by way of particular configurations and programming, the controller 102 may be a special-purpose system or device. For instance, such special-purpose system or device may include one or more dedicated processing units or modules that may be configured (e.g. hardwired, or pre-programmed) to carry out steps, in accordance with aspects of the present disclosure.

In some embodiments, as shown in FIG. 1B, the signal generator 104 may include one or more signal module(s) 108 configured to generate and provide various electrical signals (e.g. current or voltage signals) to microcoils forming μTMS elements in the microcoil assembly 106. As non-limiting examples, the signal module(s) 108 may include a Signal Generator Card (National Instruments) or a programmable generator unit (AFG3021B, Tektronix Inc.) As shown, the signal generator 104 may also include a number of amplifier module(s) 110 configured to amplify signals generated by the signal module(s) 108. By way of example, the amplifier module(s) 110 may include audio amplifiers (Pyle) or class D amplifiers, with power settings of up to 12 kW or more. In some aspects, microcoil crosstalk can be minimized by using power amplifiers for the amplifier module(s) 110. This is because power amplifiers behave like ideal current generators. Moreover, if any given amplifier module 110 is driven to overload, it may be automatically directed into a protected mode of operation, through a reset or cycling of power to off and then on.

In some aspects, the generated signals may be in the form of a pulse sequence having a plurality of pulses. The power, amplitude, duration, shape, and frequency of the pulses may selected to achieve a desired level or depth of stimulation, as well as optimize heat or magnetic forces induced in the microcoil assembly 106. By way of example, pulse durations may be in a range between approximately 5 μs to approximately 500 μs, and pulse frequencies may be in a range between 1 kHz and 1000 kHz, although other values may be possible. In some aspects, the generated pulses may be ramped-shaped pulses, such as pulses having triangular waveforms with peaks up to approximately 50 A or more. In some aspects, the rise time of such ramp-shaped or triangular pulses may be approximately 10 μs to approximately 20 μs, or longer.

Because microcoils of μTMS elements, in accordance with the present disclosure, have much lower inductance as compared conventional TMS coils, it is possible to drive them with short current pulses having arbitrary waveforms. In some implementations, pulse shape may be optimized to maximize the energy transfer to the tissue while controlling the heating of the microcoils.

As shown in FIG. 1B, in some embodiments, the signal generator 104 may optionally include a number of switching modules 112. The switching module(s) 112 may be controlled by the controller 102 and configured to direct signals to various μTMS elements in the microcoil assembly 106. For instance, in one non-limiting example, the switching module(s) 112 may include one or more solid state switches (National Instruments).

In some embodiments, the system 100 may also include one or more temperature sensor(s) 114. These may be used to provide temperature readings to the controller 102 to indicate the heating of the skin, and/or heating of the μTMS elements in the microcoil assembly 106, and/or heating of the amplifier module(s) 110. Should excessive or undesired heating occur, the controller 102 may adjust operation of the signal module(s) 104 to prevent damage to the patient or the various components in the system 100. In some situations, the controller 102 may be configured to reset affected amplifier module(s) 110 by powering them down, and/or switching operation to different amplifier module(s) 110.

As mentioned, the microcoil assembly 106 may include a number of μTMS elements arranged in any configuration. For instance, in some applications, the microcoil assembly 106 may include an array of μTMS elements, arranged in a manner to provide stimulation to specific targets in a subject's cortex. Non-limiting examples of targets in the cortex may include the primary motor cortex (M1), the primary somatosensory cortex (S1), and elsewhere. In addition, the microcoil assembly 106 may be configured to couple to the scalp of a subject and deliver stimulation using the μTMS elements. To this end, the microcoil assembly 106 may include various components or elements (e.g. a casing, a housing, a holder, an applicator, and so forth) configured to engage with the scalp of the subject, and bring μTMS elements attached thereto or incorporated therein into contact with the scalp. In some implementations, the microcoil assembly 106 may include a wearable object, such as a headband, a cap, or the like, to which the μTMS elements may be removably or permanently attached.

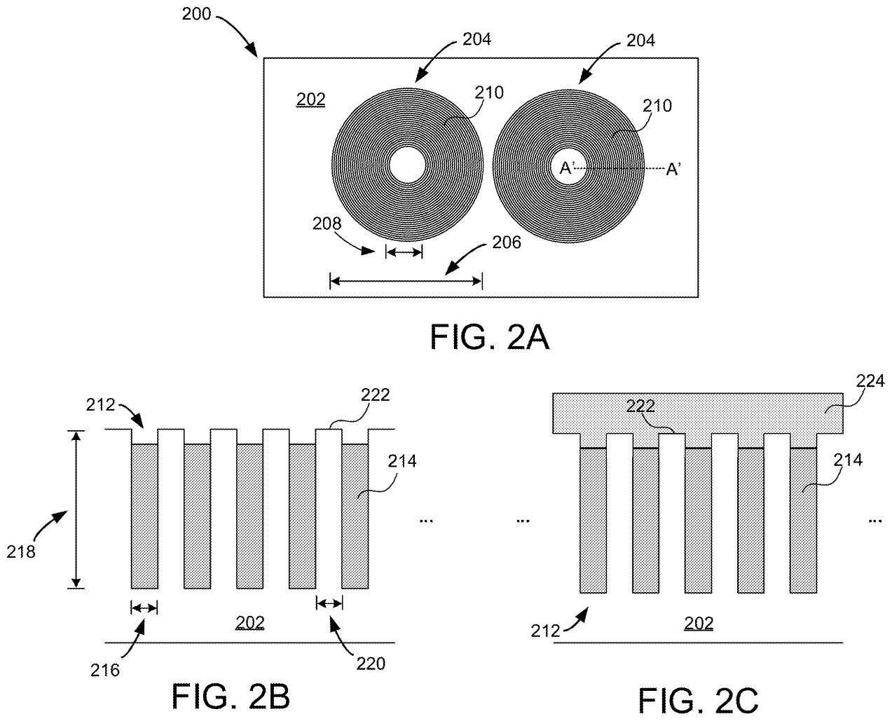

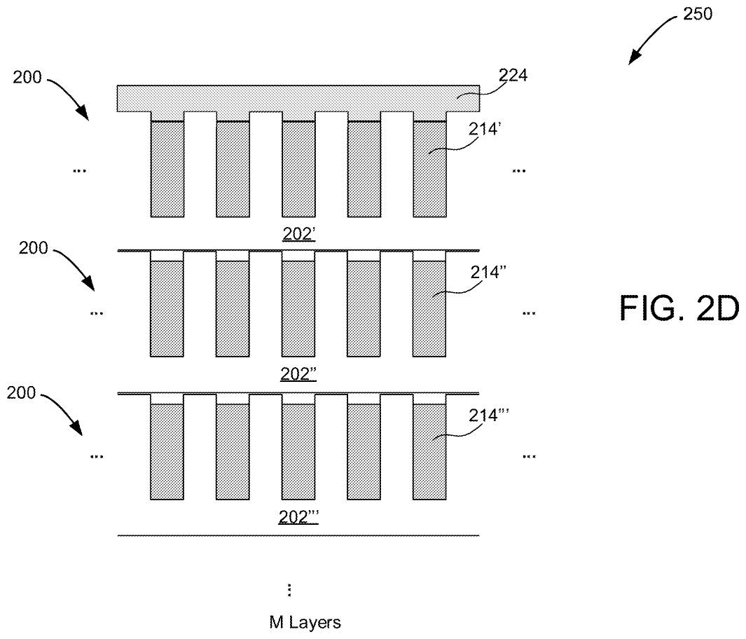

Referring specifically to FIG. 2A, the top view of an example μTMS element 200, in accordance with aspects of the present disclosure, is shown. The μTMS element 200 may include a substrate 202 having formed therein and supporting micro-coils 204. In some embodiments, the substrate 202 may be constructed using a material that is sufficiently resistive to prevent current leakage (e.g. resistivity greater than 10⁴ Ω-cm) In addition, the substrate 202 may be constructed using a material that can maintain structural and/or mechanical integrity due to forces induced by current in the μTMS element 200. In some non-limiting examples, the substrate 202 may be constructed using silicon, sapphire, plastic and similar materials. In some applications, the substrate 202 may include an elastic material, such as a polymer (e.g. polyethylene, polyamide, and so forth), and similar materials. The shape and dimension of the substrate 202 may vary depending on the particular application.

As illustrated, a pair of microcoils 204 may be arranged in a figure-of-eight configuration, although other configurations may be possible. For instance, the microcoils 204 need not be circular, and may be square or other shape. With respect to the microcoils 204 shown in FIG. 2A, each may be defined by an outer diameter 206 and inner diameter 208, and include a number of turns N of conductive micro-wire 210 or micro-traces having a curved shape. In some embodiments, the conductive micro-wire 210 of each microcoil 204 may include greater than 50 turns, and more specifically, greater than 100 turns, with lengths L up to 300 cm, or more. Other micro-trace lengths or number of turns may be possible, depending upon the desired stimulation level, and other factors. In some embodiments, the conductive micro-wire 210 of the microcoils 204 may be arranged in a double or triple interleaved spiral pattern.

Referring specifically to FIG. 2B, the cross-sectional view along line A′-A′ in FIG. 2A is illustrated. As shown, the conductive micro-wire 210 may be constructed by filling at least a portion of micro-trenches 212 formed in the substrate 202 with a conductive material 214. The micro-trenches 212 may be formed using a variety of fabrication techniques. For example, the micro-trenches 212 may be formed using photolithography and Deep Reactive Ion Etching (DIRE) technique. In some embodiments, the conductive material 214 introduced in the micro-trenches 212 may include a material with high conductivity and heat capacity, such as copper, silver, gold, or other similar material or alloy. This prevents excessive heating and damage to the μTMS element 200.

In some embodiments, the conductive material 214 may include a seed layer for improving adhesion of the bulk layer of the conductive material 214 to the surfaces of the micro-trenches 212. The seed layer may, but need not be, the same material as the bulk layer. By way of example, the seed layer may have a thickness of approximately 5 nm to approximately 200 nm. The conductive material 214 may be introduced in the micro-trenches 212 using a variety of deposition techniques, including evaporation, sputtering, chemical plating, electroplating, and so forth.

The micro-trenches 212 may be defined by a width 216 and a depth 218, and separated by a spacing 220 or pitch. By way of example, the width 216 may be in a range between approximately 1 μm to approximately 50 μm, although other widths may be possible. The depth 218 may be in a range between approximately 50 μm to approximately 700 μm, or more. In some aspects, the aspect ratio of the micro-trenches 212, defined by the depth to width ratio, may be up to 20, or more. The spacing 220 may be in a range between approximately 1 μm to approximately 50 μm, although other spacing values may be possible. In some implementations, the width 216 and depth of the micro-trenches 212, in combination with the conductive material 214 therein, may be selected to achieve a low microcoil resistance (e.g. less than approximately 5Ω, or more specifically, around 2Ω). Low coil resistance can control heating and provide compatibility to audio amplification.

In some embodiments, the top surface 222 of the substrate 202 may be covered with an encapsulation layer 224, as shown in FIG. 2C. In one non-limiting example, the encapsulation layer 224 may be constructed using silicon nitrite, although other non-conductive materials or alloys may be used. Such encapsulation layer 224 can protect the microcoils 204 from moisture and oxidation, and insulate the subject from heating produced by the microcoils 204. The encapsulation layer 224 may extend partially into the micro-trenches 214, as shown in FIG. 2C. However, this need not be so. For instance, if the conductive material 214 completely fills the micro-trenches 212, then the encapsulation layer 224 can rest on top of the conductive material 214 and top surface 222 of the substrate 202. In some aspects, the encapsulation layer 224 may include multiple layers of materials. For instance, the encapsulation layer 224 may include a first layer (e.g. a silicone conformal coating) on top of the substrate 202, and a second layer (e.g. an aerogel) on top of the first layer.

To enhance stimulation provided at any one location in a subject's cortex, in some embodiments, multiple layers may be combined together to form a multilayer stack 250, as shown in FIG. 2D. By way of non-limiting example, the multilayer stack 250 may include between approximately 20 to approximately 50 layers. The layers may be coupled together using various methods. For example, the layers may be attached using an adhesive, or using various direct bonding techniques known in the art, such as thermo-compression bonding, and others. In some embodiments, the layers may be hermetically sealed to prevent oxidation and moisture-induced degradation. In addition, as shown in FIG. 2D, the top-most layer may be covered with an encapsulation layer 224 for purposes of protection and heat isolation. In some embodiments, a number of multilayer stacks 250 may be arranged to form a stimulation array for stimulating multiple targets in a subject's cortex. As described, such stimulation array may be used for cortical mapping,

Although micro-trenches 212 of different μTMS elements 200 in the multilayer stack 250 appear to be aligned in FIG. 2D, this need not be so. This is because as the number of layers in the multilayer stack 250 increases, μTMS elements 200 further away from the subject's scalp and cortex will have a reduced effect due to geometrical and physical law considerations. To compensate for such reduced effect, the diameters of the microcoils 204 in the different μTMS elements 200 may be incrementally changed, for instance. Other modifications may also be possible. For instance, the μTMS elements 200 may be optimized to determine μTMS-tissue depth penetration and optimum coil characteristics including topology (double or single), dimensions (and quantitative relation to focality), number of turns and their geometry, as well as input power requirements, and stimulus waveform characteristics (amplitude, shape and duration).

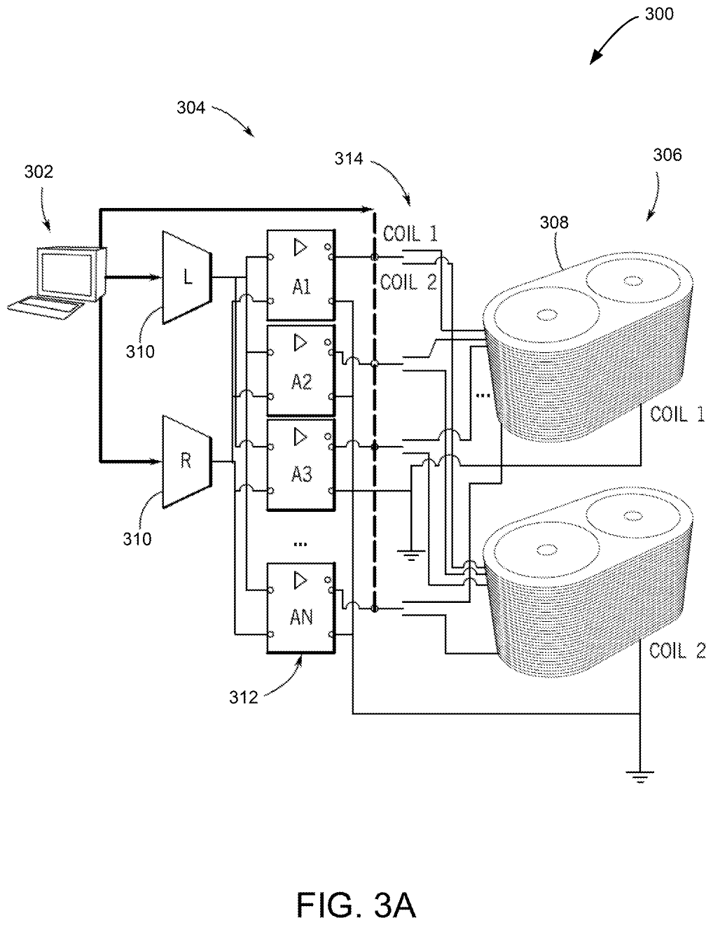

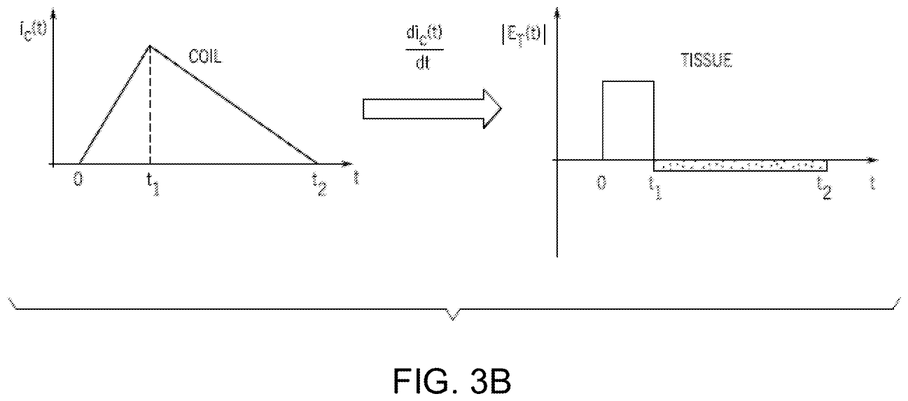

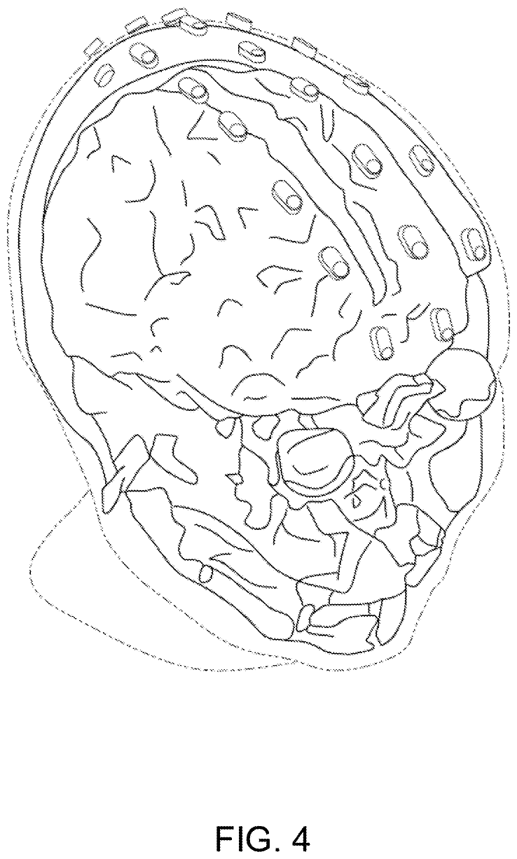

By way of example, FIG. 3A shows an example architecture a system 300, in accordance with aspects of the present disclosure. The system 300 includes a controller 302, a signal generator 304 and a microcoil assembly 306. As shown, the microcoil assembly 306 includes two multilayer stacks 308. However, the system 300 need not be limited to two multilayer stacks 308, and may include an array of multilayer stacks 308 arranged in accordance with desired a desired application (FIG. 4).

Referring to the example of FIG. 3A, the signal generator 304 includes two signal modules 310 connected to a number of amplifier modules 312. The amplifier modules 312 are also connected to a number of switching modules 314 that distribute and control the electrical signals (e.g. current pulses) provided by the amplifier modules 312. The controller 302 (e.g. a lunchbox computer lunchbox) may run software (e.g. Labview) that is configured to control the signal modules 310 (e.g. a Signal Generator card by National Instruments) to provide the electrical signals to the amplifier modules 312. In particular, the software may be configured to control the shape and amplitude, duration and train of stimulation pulses so as to deliver the optimally shaped pulses. By way of example, FIG. 3B shows that an optimally shaped pulses may be triangular pulse. This is because a triangular pulse delivered to a microcoil in the microcoil assembly 306 becomes a square pulse in tissue, as shown.

In some applications, a longer rise time may be necessary to provoke a strong stimulation. As such, the rate of change of the raising slope in the triangular pulse (FIG. 3B) may be reduced. In addition, to compensate for reduction in the magnitude of induced electric field, more layers may be added to the μTMS element. Alternatively, the number of turns in the microcoils may be increased by reducing the width of micro-traces and increasing their depth to keep the resistance low.

In some aspects, the controller 302 in FIG. 3A may be configured to deliver current pulses to microcoils in the microcoil assembly 306 using a pulse sequence that minimizes undesired heating and coil damage. As described, this may be achieved by monitoring the temperature using various temperature sensors, and thereafter estimating and controlling the sequence so that overheating may be avoided. In some implementations, the controller 302 may be configured to estimate the heating of a particular microcoil and stop the pulse delivery if the heating approaches a damaging temperature. In addition, the controller 302 may also oversee the safety of the subject being treated, making sure that pulses with known harmful effects are not delivered to the subject.

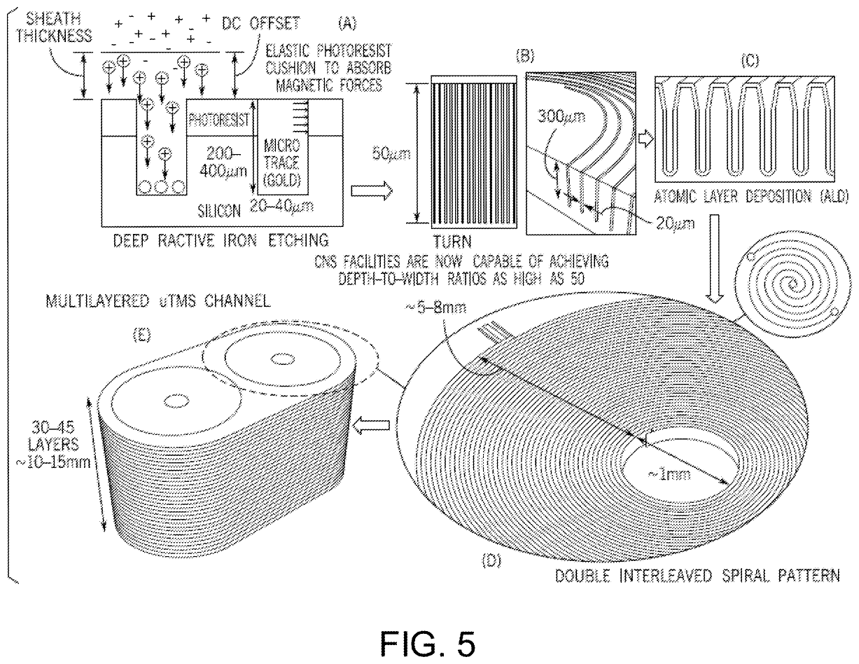

For purposes of illustration, FIG. 5 shows a non-limiting method for fabricating μTMS elements, in accordance with aspects of the present disclosure. Specifically, a photolithography technique, as shown in panel (A) is applied to create micro-trenches into a substrate. To do so, a photoresist layer is first applied to a substrate (e.g. silicon), followed by a deep reactive ion etch (DIRE) to produce the micro-trenches. In some aspects, an elastic photoresist cushion may be used to absorb magnetic forces. As shown in the figure, the micro-trenches may have high aspect ratios (up to 50 or more), using current microfabrication technologies. In the example shown, the depth (including the resist) of the micro-trenches may be up to between 400 μm, and the width may be up to 40 μm (shown in panel B), although other values may be possible. To begin the process of introducing the conducting material in the micro-trenches, a conformal deposition process, e.g. atomic layer deposition (ALD), may be used to “seed” the micro-trenches, as shown in panel (C). As an example, the micro-trenches may be seeded with a layer of gold. Then, the conductive material (e.g. gold) may be introduced, for example, using an electroplating process. In this manner, a dual- or triple-interleaved spiral μTMS element may be produced.

In one non-limiting example, as shown in panels (B) and (D) the dimensions of the microcoil forming a dual-interleaved spiral μTMS element may be: inner diameter (ID): approximately 1 mm; outer diameter (OD): approximately 12.6 mm; micro-trench width: approximately 20 μm; micro-trench depth: approximately 300 μm, and micro-trench spacing: approximately 15 μm. Furthermore, as shown in panel (E), the μTMS element may include 4-45 layers. Such dual-interleaved spiral provides a large ampere-turn (e.g. approximately 50 A in 30 layers with 50 turns), which is enough to deliver electric field excitations in tissue greater than 60 V/m. In addition, such design limits the total length (˜90 cm) of individual traces with a total trace resistance (<5Ω), which reduces heating (<8° C.) and maintains compatibility with audio amplifiers. Results of our preliminary feasibility studies and details of the fabrication and verification are given below.

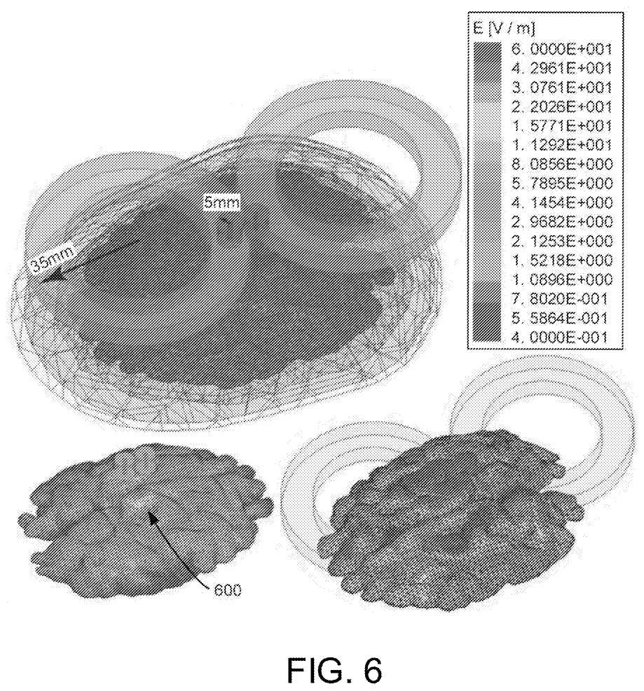

The feasibility of the present approach has been comprehensively investigated using finite element method (FEM) modeling. In a preliminary study, a highly focal activation (i.e. less than 3 mm) at the cortical tissue was achieved using a 30-layer (˜12 mm thick) μTMS element with a 5 mm outer radius and 0.5 mm inner radius (FIG. 6). A multi-compartment, anatomically realistic model of the human head developed from averaged magnetic resonance (MR) images of 20 healthy subjects was used to simulate the magnetically induced electric fields in the scalp and cortex. Simulations were implemented in ANSYS Maxwell3D (ANSYSY, Pittsburgh), solving a T-Ω formulation of the Maxwell's Equation in a full-wave eddy current analysis. A total effective equivalent (i.e. per single turn coil) current of 150 kA applied at 20 kHz produced an electric field greater than approximately 60 V/m in a focal cortical area of approximately 2 mm. Such currents have enough magnitude to elicit supra-threshold stimulation. In addition, the magnitude of induced electric fields in the skin of the scalp remained below 600 V/m, which is the maximum accessible field of conventional TMS coils, and thus, is unlikely to provoke painful stimulation in the scalp.

For purposes of comparison, simulations were also carried out for a state-of-the-art commercial TMS coil (r_{c}=35 mm). The results are shown in FIG. 6. As appreciated from the figure, the focality obtained using a μTMS element (bottom-left) is significantly enhanced compared to the commercial TMS coil (bottom-right). This is apparent from the electric field distributions in the cortex. In particular, for the μTMS element, values of high electric fields are concentrated in a small focal area of the cortex, as indicated by arrow 600. By contrast, high electric fields are distributed throughout the cortex for the commercial TMS coil.

The temperature rise in a μTMS element, according to the present disclosure, may be calculated using the following equation:

ΔT=η(cρA²)⁻¹∫₀^{Δt}1²(t)dt

For a single excitation pulse with maximum current of 50 A, and η=1.68×10⁻⁸ Ωm, ρ=8,700 kg/m³, c=387 J/kg° C., and A=6×10⁻⁹ m²), the temperature change is below 8° C. It is expected that such temperature changes may be tolerated.

In addition, Lorentz forces between micro-traces of adjacent layers in a μTMS element may be calculated according to: F(t)=∫∫∫J(t)×B(t)dVol. Although pressure forces computed were not negligible (˜10 kPa pressure applied on micro-trench walls), it is unlikely that the silicon substrate would crack under a pulse per second, as suggested herein. Moreover, this type of mechanical impact on Si has been previously studied and the estimated pressure forces are well below the strike level impact force that would produce micro fractures.

In practice, it is contemplated that a lower electric field threshold will be sufficient to provoke cortical stimulation using μTMS elements, as described. This prediction is based on recent experiments with micro-magnetic stimulators measuring the trans-synaptic activation of neurons in the inferior colliculus in response to μMS stimulation of the dorsal cochlear nucleus in anesthetized hamsters. Interestingly, results of both, computational modeling and physiological experiments, demonstrated that microcoils are capable of provoking action potentials with induced electric fields having amplitudes well below those required in conventional electric and magnetic stimulation (<10 V/m). One explanatory hypothesis is that because microcoils induce electric fields with a much higher spatial gradient (due to their small size), and because the neural activation function depends on both the magnitude of the induced electric field and the magnitude of the electric field's derivative along the neuron's axes, microcoils are capable of eliciting action potential at lower field thresholds.

Similarly, it is also contemplated that the μTMS elements envisioned herein will be capable of inducing transcranial cortical activation at electric field thresholds below those of large conventional TMS coils. This is based on numerical simulations performed, which show that even at a depth of 15 mm below the coil's surface, μTMS elements may still induce a higher electric field gradient compared to conventional TMS coils (maximum of 120V/m² vs. 40 V/m²). To date, the preliminary studies have confirmed the feasibility of developing significantly more focal coils with highly reduced dimensions. It is contemplated that a quantitative relationship between the area of cortical stimulation and dimension of μTMS elements exists. Different topologies (e.g., single vs double coils) with a range of different dimensions (5 mm-15 mm outer diameter) are contemplated herein. In addition, several optimum designs may be identified for fabrication. For each design, alternative solutions may be devised to account for potential complications (e.g., fabrication constraints, excessive heating, and so forth). In addition, the optimum number of layers in each μTMS element may be determined. Furthermore, optimum pulse shapes may also be identified to maximize neural activation. It is contemplated that an integrated approach combining FEM field calculation with neuron cable modeling is possible.

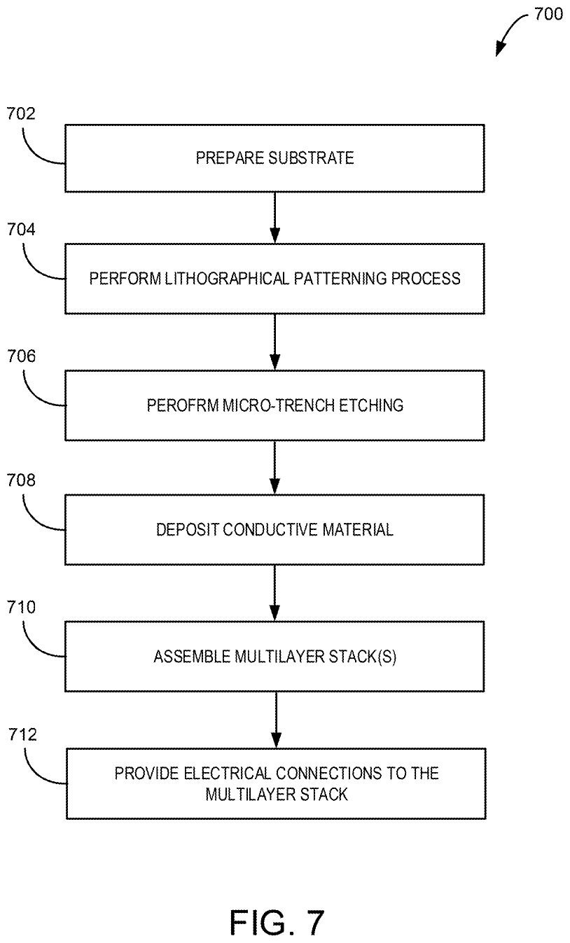

Turning now to FIG. 7, the step of a process 700 for manufacturing μTMS elements, in accordance with aspects of the present disclosure are shown. The process 700 may begin at process block 702 with substrate preparation. Preferably, the substrate may be an undoped intrinsic nonconductive substrate with a high material resistivity (>10⁴ Ω-cm), which would prevent current leakage between micro-traces. In one non-limiting example, the substrate may be a silicon wafer having, for example, a 150 mm diameter although larger or smaller diameters may be possible. In particular, larger diameter wafers allow more microcoils to be produced at once. In some aspects, preparation at process block 702 may include various plasma or RCE cleaning steps. In addition, the substrate may be vacuum-coated with the adhesion promoter, such as Hexa-Methyl-Di-Silazane (HMDS), which transforms the surface of the substrate from hydrophilic to hydrophobic by replacing a hydroxyl group with a trimethylsilyl group.

Then, at process block 704, a lithographical patterning may be formed on the substrate. To do so, the substrate may be spin coated with a photoresist layer (e.g. positive SPR220 photoresist). As an example, the thickness of the photoresist layer may vary between approximately 5 μm and 25 μm, although other values may be possible. A pre-bake (softbake) may then be performed with, say, a 30 second ramp up to 115° C. for a minimum of 90 seconds in a hotplate.

Photolithography may then be carried out to pattern the photoresist layer. For example, direct mask writers (Heidelberg DWL66 or MW-1 systems) may be used. The timing of exposure (dose effect on traces), prebaking for removal of defects (border scalloping), and lift-off resist (LOR) may vary depending upon photoresist. Finally, a post-bake (e.g. a hard bake) may be applied to stabilize/harden photoresist that cannot be removed, to improve adhesion with the substrate, and to remove any residual solvent and developer.

A micro-trench etching may then be performed, as indicated by process block 706. For instance, in one non-limiting example, a deep Rapier reactive-ion etch (DRIE) technique may be used to etch the micro-trenches in the silicon using the SPTS Rapier etcher. DRIE can be especially advantageous due to anisotropic etching that allows fabrication of very high and steep trace walls (e.g., 300 μm) in narrow micro-trench widths (e.g., 20 μm). Once residues of the chemical etching are removed, the substrate may be laser drilled to provide a vertical ground electrical connection.

Then, at process block 708, a conductive material may be deposited. This step may include first performing a conformal coating to add a seed layer to the etched micro-trenches. For example, chemical vapor deposition or physical vapor deposition may be used to maximize shadowing or step coverage and achieve maximal conformal deposition. In some aspects, conductive material (e.g. gold) may be evaporated under different angles without breaking the vacuum. This is a useful step that allows the deposition of the seed layer in a very conformal manner, and without obstructing the narrow micro-trenches produced by the etching step. In some aspects, an XRR—X-Ray Reflectometry technique may be used to measure the thickness of seed layer, which can be at least 10 μm thick. A bulk layer deposition of the conductive material may then be performed. For instance, the conductive material, or portions thereof, may be deposited using an electroplating process, an atomic layer deposition process, other others. In particular, the electroplating process may be performed using an electroplating solution (e.g. a gold solution) from a commercially available electroplating kit, or by outsourcing (Metrigraphics LLC, Lowell, Mass.). In some implementations, the bulk layer may reach a thickness of up to 200 μm, or more.

In some aspects, a metrology step may be carried out at process block 708. For instance, profilometers, stress measurement, ellipsometry, and sheet resistance mapping may be utilized. The last technique is a useful tool because the microcoils are designed to carry a large amount of current for a very short period of time. Metrology may be used validate the fabrication process.

An assembly of one or more multilayer stacks may be performed, as indicated by process block 710. For example, multilayer stacks having approximately 4 to 45 layers may be assembled using thermocompression bonding, or other technique. As described, such bonding can help protect the microcoils from moisture and oxidation, while insulating the subject from heating and ensuring a mechanical stability and a hermetically-sealed encapsulation. As described, the top layer(s) of the multilayer stacks may be encapsulated with an encapsulation layer (e.g. silicon nitrate).

And finally, at process block 712, electrical connections may be provided to the multilayer stack(s). To this end, various wire bonding techniques may be utilized. For example, microcoil wiring, exposed by the previous micro-trench etching process, and may be wire bound to external electrical connections, such as the amplifier and switching modules described with reference to FIGS. 1B and 3A. Alternatively, or additionally, the electrical connections may be made to pads on a holder or housing that retains the multilayer stack(s). Such pads may have solderable pins, or other electrical couplings, to provide electrical connection to external devices or modules. As such, a holder may be designed and 3D printed, for example, to provide electrical connection of microcoils in the multilayer stack(s) to the amplifier modules, and to allow safe handling.

It is possible that the μMTS element geometries may be optimized for maximum current and thus maximum magnetic field flux in the tissue vibrate due to the large Lorentz forces may ultimately crack with long reverberating pulses (although not likely with the initial single pulse experiments). In that case, the thickness of the photoresist layer may be increased to allow for the absorption of the vibration during current pulsing. Additionally, or alternatively, a polymeric substrate that is elastic, such as polyethylene or polyamide, may be utilized. If the resistivity of the microcoils in the μMTS element is too high, the number turns can be reduced and the cross section increased. Also, additional layers may be added to compensate. Furthermore, copper electroplating may be used instead of gold, while keeping a thin top layer of protective gold.

It is contemplated that μTMS elements of the present disclosure may be used on human and animal subjects. In particular, human experiments are envisioned to evaluate the safety and focality of μTMS coils for motor cortical output mapping and demonstrate the feasibility of using an array of μTMS elements to stimulate both sensory and motor cortex representations of single digits.

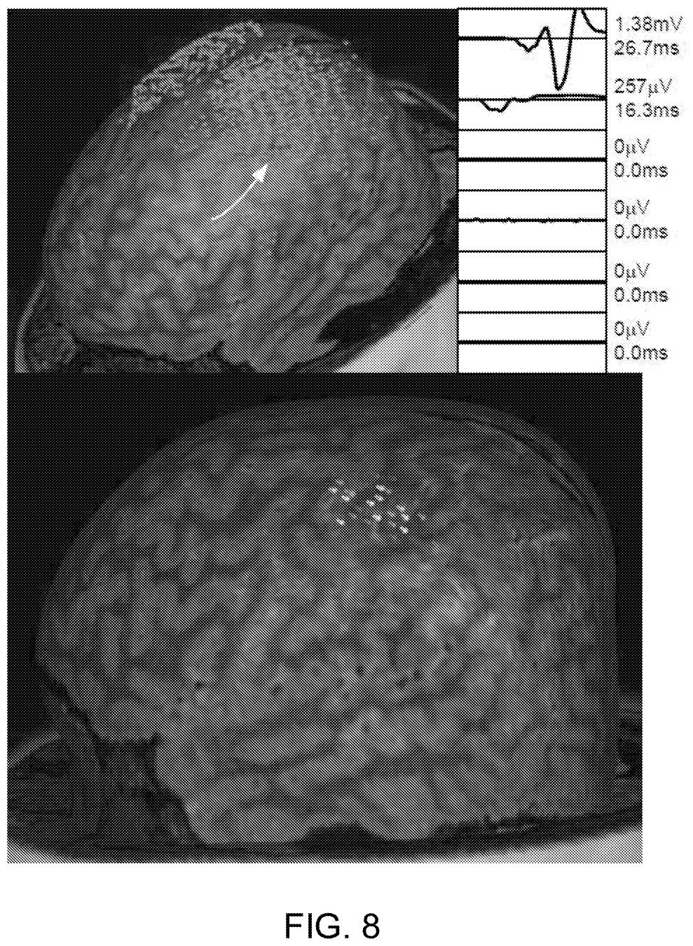

By way of example, FIG. 8 shows example MR images and evoked potentials from an applied stimulation. In particular, the image on the top is a three-dimensional representation of a subject's brain derived from a standard clinical MRI using NexStim software. Each orange pin represents the location where a single pulse of TMS was applied during a mapping procedure. The electromyography (EMG) data on the top right shows a sample motor evoked potential in the abductor pollicus brevis (APB) (top EMG lead, in green) from a single TMS pulse applied at the location identified by the arrow in the top panel. The image on the bottom shows all locations in which the TMS pulse resulted in motor evoked potentials (MEPs) of the right APB. The color scale denotes MEP amplitude, with white pins in and around the ‘hand knob’ region of the motor cortex resulting in the strongest response.

As described, the systems and methods of the disclosure may be used for cortical output mapping. To this end, one or more μTMS elements may be used to stimulate multiple locations around the subject's cortex. In some aspects, MEPs induced in the four intrinsic hand muscles (APB) flexor dorsal interosseous (FDI), adductor digiti minimi (ADM), and first lumbrical interosseous (FLI)) may be recorded. Referring to FIG. 9, example cortical output maps of the FDI, APB, and ADM muscles in two representative subjects are shown. Current direction and topographic information are indicated on the figure. The angle of measurement from the central sulcus of both hemispheres is shown. The dotted blue line shows the central sulcus. Note the minimal separation between the optimal targets for maximal activation of each target muscle. In some aspects, the cortical output maps may also be quantified. As such, optimal cortical target and centers of gravity may be calculated. In addition, the degree of coactivation of multiple muscles may be computed, which is expected to be greater than the coactivation measured using previous technologies.

In accordance with aspects of the present disclosure, above described μTMS elements may be used to provide stimulation to the motor cortex. Specifically, if no reasons for subject exclusion are identified, the following experimental steps may be completed: 1. Stimulation of the motor cortex to evoke MEPs from APB, FDI or ADM of the hand contralateral to the stimulation. 2. Stimulation of sensory cortex to block detection of electric stimuli to the tips of thumb, index or little finger from intrinsic hand muscles of the hand contralateral to the stimulation. 3. Paired-pulse stimulation with stimulation of sensory cortex preceding stimulation of the motor cortex at a variable interval to evaluate cortico-cortical interactions between sensory and motor cortices.

In addition, stimulation of sensory cortex, in accordance with aspects of the present disclosure, may also be used to block detection of electric stimuli to the tips of thumb, index or little finger from intrinsic hand muscles of the hand contralateral to the stimulation. To this end, stimulation may be delivered to the primary somatosensory cortex, as defined neuroanatomically 20 msec after an electrical stimulus is applied to the contralateral middle finger. Randomly, 50% of the trials may involve a TMS pulse and separately 50% of the trials may involve an electrical sensory stimulus delivered at 2.5 times sensory threshold intensity, as determined by the method of limits, through a pair of surface electrodes applied to the finger pad, separated by 1.5 cm, for example. The accuracy of the participant (# true positives+# true negatives/total trials) may then be recorded. Once an attenuation is found, analysis of the neighboring fingers may then be performed to determine the focality of the μTMS stimulation. The focality will be contrasted with a standard Magstim, figure-of-eight coil.

Paired-pulse stimulation with stimulation of sensory cortex preceding stimulation of motor cortex at a variable interval may also be performed to evaluate cortico-cortical interactions between sensory and motor cortices. Using an array of μTMS elements positioned at least over both S1 and M1, the M1 location for maximal FDI stimulation, representing the index finger may be located, along with the S1 location in which maximal somatosensory attenuation of the index finger is found. With both sites located, cortical-cortical inhibition may be tested via pulses to S1 preceding pulses to M1 at varying intervals from 5 msec to 20 msec, for example. MEPs may be recorded with varying delays between S1 and M1 to determine the chronometry of the putative cortical-cortical inhibition.

In some applications, it may be advantageous to ensure that there is no “cross-talk” between stimulation over S1 and over M1, for instance. That is, if stimulation over S1 causes MEPs, then it may not be possible to disentangle the direct effect of spread of the stimulation from S1 with the indirect, transcortical effect desired for testing. If this occurs, the intensity of stimulation of the S1 may be lowered. Additionally, or alternatively, a frameless stereotaxy may be used for repositioning (e.g. slightly posteriorly) to prevent the degree of spread.

The safety of stimulation performed using μTMS elements described herein may be verified using functional measures, as well as sensitive MRI sequences to ensure there are no adverse effects. While the basic principles of magnetic induction are similar between μTMS and TMS, the present approach is the first of its kind in human study. As such, additional safety measures may be warranted. Among the various safety evaluations based on various baseline measurements, functional measures of strength and dexterity and MRI may be performed before and after μTMS. For instance, both grip strength and thumb-little finger apposition may be measured with dynamometry using specifically designed equipment. By way of example, five measures of each action may be performed before and immediately after μTMS. If a decrease 20% is observed, testing may be repeated at additional time points (T=30 min, 60 min, etc) and persisting weakness may be deemed a serious adverse event, triggering further evaluation. In addition to strength, dexterity may also be measured with the Grooved Pegboard Test. Again, any decrement of 20% or greater may trigger additional testing and any persistent impairment at T=60 minutes, for instance, may be deemed a serious adverse event.

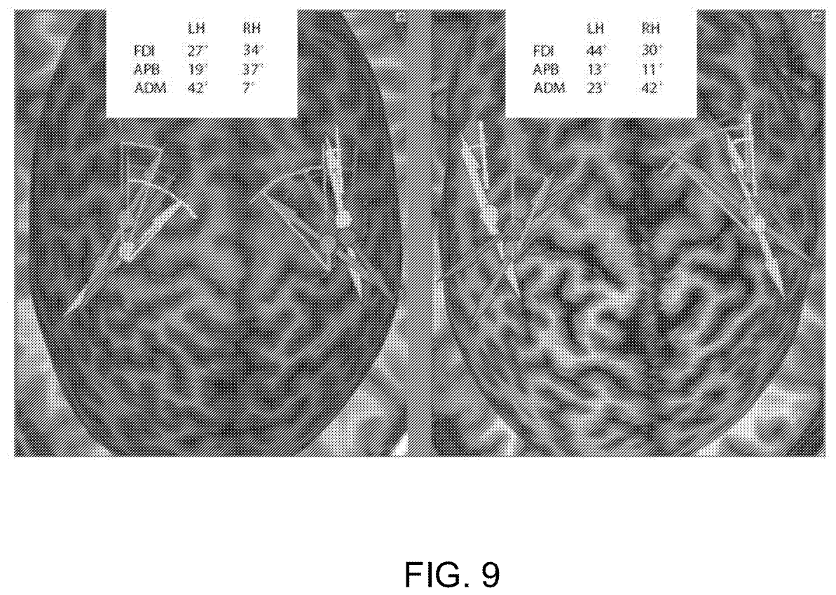

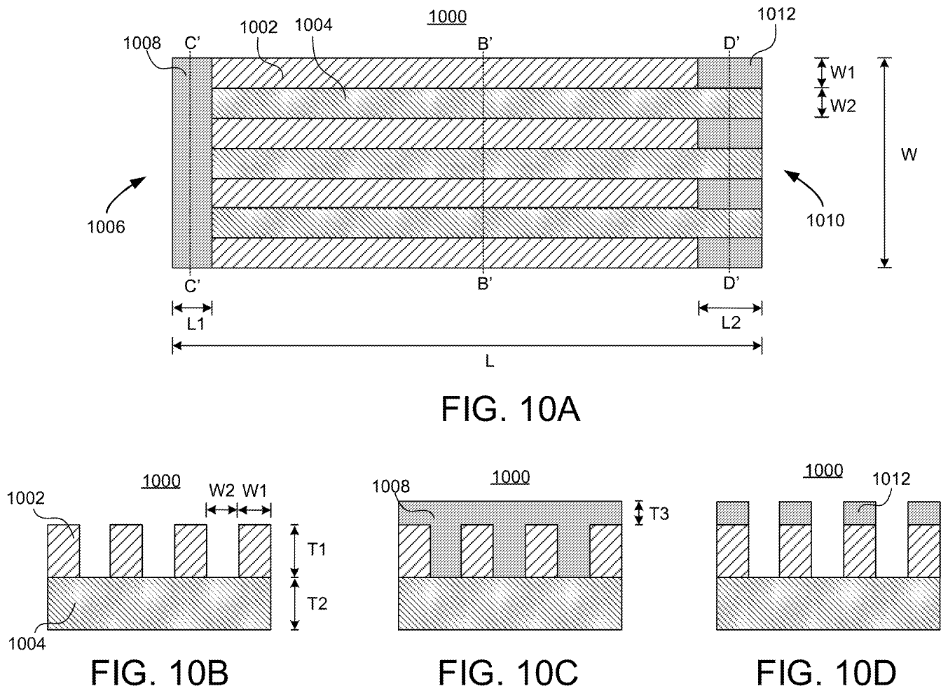

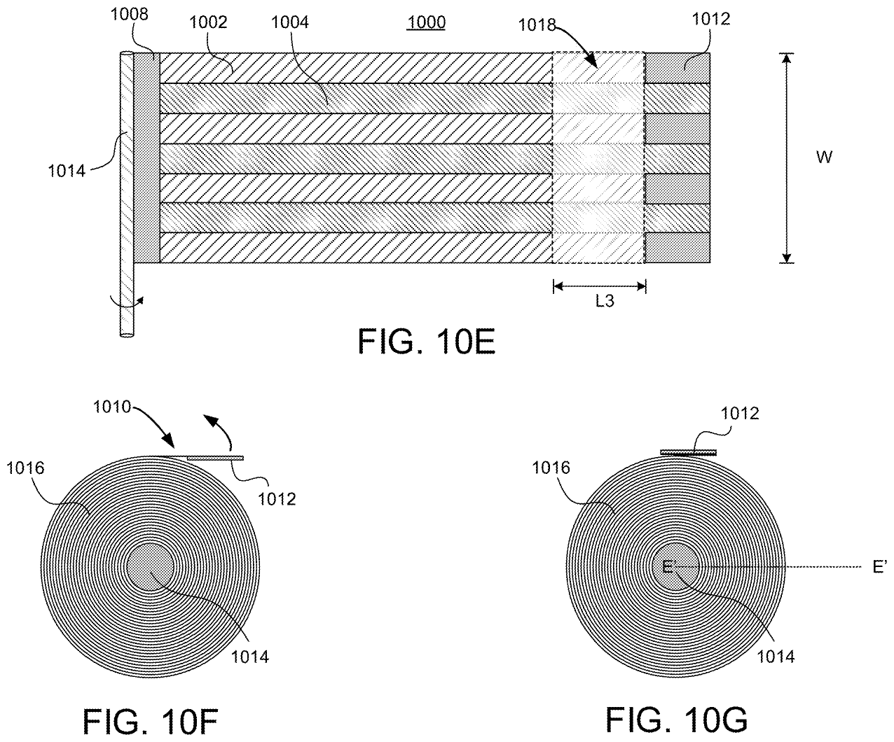

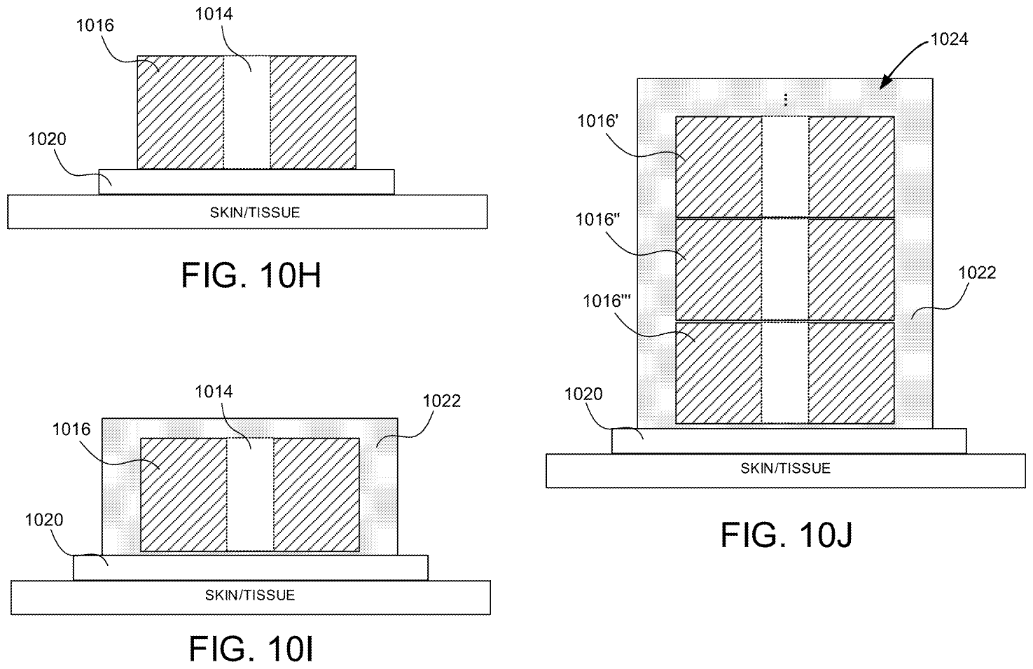

Referring now to FIGS. 10A-10G, another embodiment of a μTMS element 1000 is described. With particular reference to FIG. 10A, the μTMS element 1000 may include a number of conductive micro-wires 1002 or micro-traces formed using a conductive material (e.g. copper). The conductive micro-wires 1002 may be shaped as parallel stripes with width W1 between approximately 50 μm to approximately 1000 μm, thickness T1 between approximately 5 μm to approximately 50 μm, and length L between approximately 1 m to approximately 4 m, although other values may be possible. As illustrated in the cross-sectional view along line B′-B′ (shown in FIG. 10B), the conductive micro-wires 1002 are supported by a substrate 1004, which in some embodiments, is a flexible substrate (e.g. a polyamide substrate, a Kapton substrate, and so on). The separation or width W2 of space between the conductive micro-wires 1002 may be between approximately 50 μm to approximately 1000 μm, although other values may be possible. In addition, the thickness T2 of the substrate 1004 may be in between approximately 10 μm to approximately 50 μm, although other values may be possible.

At the first end 1006 of the μTMS element 1000 is a grounding pad 1008 that spans the width W of the μTMS element 1000 and extends a length L1 along the length L of the μTMS element 1000. As shown in the cross-sectional view along line C′-C′ (FIG. 10C), the grounding pad 1008 electrically shorts the conductive micro-wires 1002. By contrast, at the second end 1010 of the μTMS element 1000 are contact pads 1012, which provide individual electrical contact to each of the conductive micro-wires 1002 or micro-traces, as illustrated in the cross-sectional view along line D′-D′ (shown in FIG. 10D). The contact pads 1012 span the width W1 of the conductive micro-wires 1002 or micro-traces, and extend a length L2 along the length L of the μTMS element 1000. Electrical contact to the contact pads 1012 may be made using a variety of methods, including soldering, bonding, and others. The lengths L1 and L2 may be in a range between approximately 1 mm to approximately 4 mm, although other values may be possible. The grounding pad 1008 and contact pads 1012 are constructed using a conductive material of thickness T3. By way of example, the thickness T3 of the conductive material may be in a range of approximately between 50 to 200 μm, formed using an electroless metal immersion (e.g. nickel) and metal electroplating (e.g. gold).

Referring particularly to FIGS. 10E-10F, the μTMS element 1000 is rolled around a conductive rod 1014 to create microcoils 1016 capable of stimulation. The diameter of the conductive rod 1014 may be approximately between 0.5 mm to 3 mm, although other values may be possible. The diameter of the microcoils 1016 may depend on the length L of the μTMS element 100. The conductive rod 104 may be constructed using a conductive material such as brass or bronze, or another suitable material. In some embodiments, the conductive rod 1014 may be a threated rod or a high permeability core (e.g. ferrite). Through the rolling process, between approximately 100 to approximately 300 turns N may be generated. To maintain the shape of the microcoils 1018 following the rolling process, an adhesive layer 1018 of length L3 (e.g. between approximately 1 to approximately 5 mm) and width W may be applied to the μTMS element 1000, as shown in FIG. 10E. In some implementations, the adhesive layer may include a double sided tape.

Electrical contact to the microcoils 1016 may be made by connecting to the conductive rode 1014 and the contact pads 1012. For convenience or ease of connection, the second end 1010 of the μTMS element 1000 may be bent to expose the contact pads 1012, as illustrated in FIGS. 10F and 10G.

In one non-limiting example, the μTMS element 1000 was constructed using Flex circuit technology (e.g. FELIOS R-F775, Panasonic), and included four conductive micro-wires 1002, each with a width W1 of 711 μm, length L of 2.847 m, and thickness T1 of 35 μm, on approximately 25 μm of polyamide substrate. These dimensions were selected to produce an approximate 2Ω resistance and 70 μH inductance on each 1 oz copper micro-wire 1002. The microcoil produced had approximately 123 turns. As described above, it may be appreciated that the dimensions, number of conductive micro-wires 1002, and number of turns N may vary, depending upon the particular application desired.

In some embodiments, microcoils 1016, generated as described above, may be covered, at least in part, with one or more thermal insulators to provide protection to the subject's skin or tissue being treated, as shown in FIGS. 10H-10J. For instance, in some embodiments, a thermally insulating layer 1020 may be positioned between a subject's skin or tissue and the microcoils 1016, as shown in the figures. By way of example, the thermally insulating layer 1020 may include an aerogel material, although other materials may also be utilized. Specifically, aerogels are a particular class of porous, solid and dry materials (unlike ordinary gels), and have unique and extraordinary thermal insulation properties. Specifically, aerogels have the same solid structure of a wet gel, but there is a gas or vacuum in its pores, rather than liquid molecules. This structure allows aerogels to possess the lowest density ever been synthesized for a solid material. Furthermore, aerogels are open-porous (that is, the gas in the aerogel is not trapped inside solid pockets) and have pores in the range of less than 1 to 100 μm in diameter, usually <20 μm. In some aspects, a silica aerogel may be used, and made lighter than air by evacuating the air out of its pores. Advantageously, silica aerogels are also poor conductors of heat, and is dry, low-density, porous and in the solid state.

In some alternative designs, as shown in FIG. 10I, the microcoils 1016 may be further encapsulated in an encapsulation layer 1022 (e.g. a thermally conductive, non-electrically conductive epoxy). Furthermore, in yet another embodiment, the microcoils 1026 may be assembled into a multilayer stack 1024, as illustrated in FIG. 10J. Although shown in FIG. 10J to include an encapsulation layer 1022 and a thermally insulating layer 1020, in some implementations, the encapsulation layer 1022 need not be present.

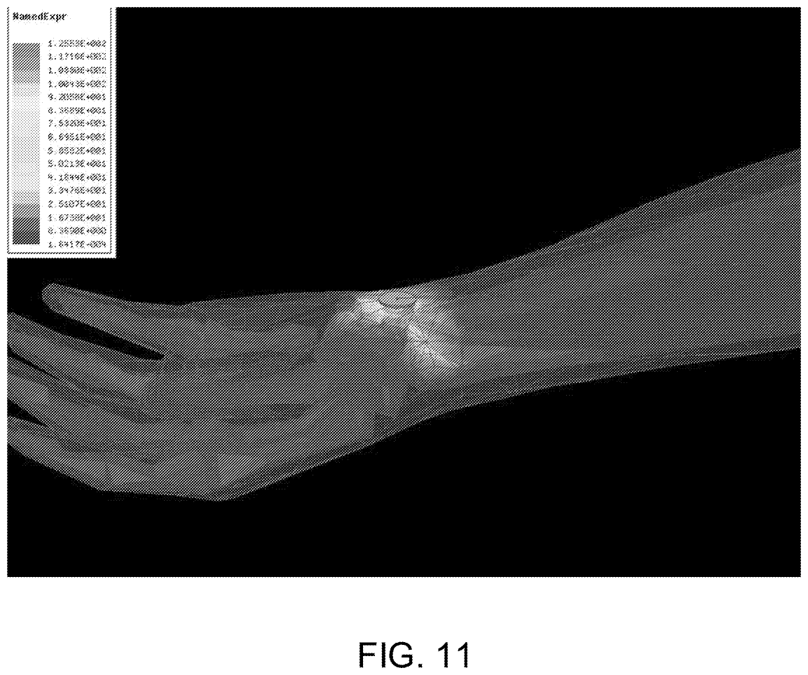

As described, the present disclosure provides for a variety of applications, including stimulation of a subject's central or peripheral nervous system. By way of example, FIG. 11 depicts an image showing electric fields produced on a human extremity using microcoils, in accordance with aspects of the present disclosure. The simulations, obtained using Finite Elements Method, illustrate a high focality of electric field amplitudes at the proximity of the medial nerve in the wrist region.

The present invention has been described in terms of some preferred embodiments, and it should be appreciated that equivalents, alternatives, variations, and modifications, aside from those expressly stated, are possible and within the scope of the invention.

The following listing of the claims replaces all previous listings:

1. A micro-transcranial magnetic stimulation (μTMS) element for stimulating a subject, the μTMS element comprising:
a substrate; and
supported by the substrate, microcoils formed using conductive micro-wires having a curved shape and capable of sustaining an electrical signal,
wherein the microcoils are configured to produce a focality of stimulation of less than 10 mm when subjected to the electrical signal.

2. The μTMS element of claim 1, wherein the curved shape is a circular shape.

3. The μTMS element of claim 1, wherein the microcoils have at least 50 turns.

4. The μTMS element of claim 1, wherein the substrate is a silicon substrate or a polyamide substrate.

5. The μTMS element of claim 1, wherein the substrate has micro-trenches formed therein, and a conductive material filling at least a portion of the micro-trenches, the micro-trenches having a width in a range between approximately 1 μm to approximately 50 μm, and a depth in a range between approximately 50 μm to approximately 5 mm.

6. The μTMS element of claim 5, wherein at least one of:
the micro-trenches have an aspect ratio defined by a depth to width ratio that is at least 20;
the micro-trenches have a spacing in a range between approximately 1 μm to approximately 50 μm; or
the μTMS element is a multilayer stack formed using multiple layers of the substrate and conductive material.

7. (canceled)

8. (canceled)

9. The μTMS element of claim 6, wherein the multilayer stack comprises between 4 and 45 layers.

10. The μTMS element of claim 1, wherein the μTMS element further comprises an encapsulation later covering at least one surface of the substrate.

11. The μTMS element of claim 1, wherein the encapsulation layer comprises an aerogel.

12. The μTMS element of claim 1, wherein the curved shape is produced by rolling the substrate around a conductive rod.

13. A system for micro-transcranial magnetic stimulation (μTMS), the system comprising:
a microcoil assembly comprising at least one μTMS element having a focality of stimulation of less than 10 mm;
a signal generator in electrical communication with the microcoil assembly and configured to provide electrical signals to the at least one μTMS element; and
a controller configured to control the signal generator to deliver the electrical signals to the at least one μTMS element,
wherein the at least one μTMS element comprises a substrate and, supported by the substrate, microcoils formed using conductive micro-wires having a curved shape and capable of sustaining the electrical signal.

14. The system of claim 13, wherein the signal generator further comprises one or more signal modules configured to generate the electrical signal.

15. The system of claim 14, wherein the signal generator further comprises one or more amplifier modules configured to amplify the electrical signal generated by the one or more signal modules.

16. The system of claim 14, wherein the signal generator further comprises one or more switching modules configured to direct the electrical signals to the microcoil assembly.

17. The system of claim 13, wherein the controller is further configured to direct the signal generator to generate the electrical signal as a sequence of pulses.

18. The system of claim 17, wherein the sequence of pulses comprises triangular pulses.

19. The system of claim 13, wherein the microcoil assembly further comprises an array of μTMS elements configured to stimulate multiple locations about the subject.

20. The system of claim 13, wherein the substrate has formed therein micro-trenches, and a conductive material filling at least a portion of the micro-trenches that form the microcoils, wherein the micro-trenches have a width in a range between approximately 1 μm to approximately 50 μm, a depth in a range between approximately 50 μm to approximately 5 mm, and a spacing in a range between approximately 1 μm to approximately 50 μm.

21. The system of claim 20, wherein at least one of:
the micro-trenches have an aspect ratio defined by a depth to width ratio that is at least 20; or
the at least one μTMS element is a multilayer stack formed using multiple layers of the substrate and conductive material.

22. (canceled)

23. The system of claim 21, wherein the multilayer stack comprises between 4 and 45 layers.

24. A method for stimulating a subject, the method comprising:
directing a signal generator, using a controller, to provide an electrical signal to a microcoil assembly having at least one μTMS element comprising:
a substrate and,
supported by the substrate, microcoils formed using conductive micro-wires having a curved shape and capable of sustaining the electrical signal,
wherein the at least one μTMS element has a focality of stimulation of less than 10 mm when subjected to the electrical signal.
